(19) United States
(12) Patent Application Publication
(10) Pub. No.: US 2025/0090369 A1
(43) Pub. Date: Mar. 20, 2025

(54) Thermally Regulated Patient Transfer Board

(71) Applicant: Costello Prosthetic Warmers LLC, Liverpool, NY (US)

(72) Inventor: Bryan Costello, Liverpool, NY (US)

(73) Assignee: Costello Prosthetic Warmers LLC, Liverpool, NY (US)

(21) Appl. No.: 18/829,402

(22) Filed: Sep. 10, 2024

Related U.S. Application Data
(60) Provisional application No. 63/582,603, filed on Sep. 14, 2023.

(51) Int. Cl. A61F 7/00; A61G 1/048

(52) U.S. Cl. CPC A61F 7/0085; A61G 1/048; A61F 2007/0054; A61F 2007/0074; A61F 2007/0088

(57) ABSTRACT

A board for thermally regulating the temperature of a patient disposed on the board is provided. The board includes a board body formed by at least two laminate layers and a temperature regulating apparatus embedded between the first laminate layer and the second laminate layer of the board body. The temperature regulating apparatus may include a heating element that selectively emits thermal radiation to at least one of the first laminate layer and the second laminate layer. Alternatively, the temperature regulating apparatus may include a fluid tube for receiving a flow of a temperature regulating fluid therethrough. The board may further include a fluid pump and a fluid tank that holds the temperature regulating fluid therein. The fluid pump generates the flow of the temperature regulating fluid though the fluid tube. The fluid tank, fluid pump, and fluid tube form a recirculating flow circuit for the temperature regulating fluid.

### CROSS-REFERENCE TO RELATED APPLICATIONS

This application claims to the benefit of U.S. Provisional Application No. 63/582,603 filed Sep. 14, 2023, which is hereby incorporated herein by reference.

### FIELD OF THE INVENTION

This invention relates generally to patient transfer boards and, more particularly, to a patient transfer board with an incorporated heating/cooling apparatus to selectively regulate a patient's temperature.

### BACKGROUND OF THE INVENTION

Maintaining an acceptable body temperature is essential for the proper functioning of essential biological systems. If the body temperature is not maintained within an acceptable range, such as during a hypothermic event, the body's ability to provide blood clotting and result in organ failure, coma, or death. Therefore, thermal regulation of a patient's temperature following a medical emergency or traumatic event directly impacts patient outcomes as well as the associated hospital costs incurred during the treatment and rehabilitation of the patient. Current temperature management regimes utilize inefficient mechanical means, such as thermal regulating garments or blankets, or invasive medical treatments that may involve inflicting further trauma to the patient.

Therefore, it would be desirable to have a system and method for thermally regulating a patient's temperature following a medical emergency or trauma experience to improve treatment outcomes. The present invention satisfies these, as well as other, needs.

### BRIEF SUMMARY OF THE INVENTION

In accordance with an aspect of the present invention, one exemplary embodiment may be directed towards a board for thermally regulating the temperature of a patient including a board body formed by at least two laminate layers and a temperature regulating apparatus embedded between the first laminate layer and second laminate layer of the board body. The temperature regulating apparatus may include a heating element that selectively emits thermal radiation to at least one of the first laminate layer and second laminate layer. Alternatively, the temperature regulating apparatus may include a fluid tube to receive a flow of a temperature regulating fluid therethrough. The second laminate layer may define a recess dimensioned to receive the temperature regulating apparatus therein.

In accordance with a further aspect of the present invention, the board may further comprise a fluid pump and a fluid tank configured to hold the temperature regulating fluid therein. The fluid pump is configured to generate the flow of the temperature regulating fluid though the fluid tube. The fluid tank, fluid pump and fluid tube may form a recirculating flow circuit for the temperature regulating fluid.

The heating element may emit thermal radiation when supplied with an electric current. The heating element may include a resistive material. The resistive material may include a resistive ink. The heating element may include a power coupling. The board may selectively and conductively couple to a power source via the power coupling. In accordance with other aspects of the present invention, the board may further include an intermediate layer positioned between the first laminate layer and the second laminate layer. A power source may conductively couple to the heating element. The heating element and power source may be positioned upon or within the intermediate layer.

The board body may include a plurality of handholds that are each peripherally positioned about the board body. The plurality of handholds may each traverse the board body. The temperature regulating apparatus may include a substrate. The heating element may be positioned upon or within the substrate.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

The accompanying drawings form a part of this specification and are to be read in conjunction therewith, wherein like reference numerals are employed to indicate like parts in the various views, and wherein:

FIG. 1 is a perspective view of an exemplary patient transfer board including an embodiment of a heating apparatus in accordance with an aspect of the present invention;

FIG. 2 is a top plane view of the exemplary patient transfer board shown in FIG. 1 with a portion removed;

FIG. 3 is a cross sectional view of the exemplary patient transfer board taken generally along line 3-3 in FIG. 2;

FIG. 4 is an exploded view of the exemplary patient transfer board shown in FIG. 1;

FIG. 5 is a perspective view of an alternative exemplary patient transfer board including an embodiment of a heating/cooling apparatus in accordance with an aspect of the present invention;

FIG. 6 is a top plane view of the alternative exemplary patient transfer board shown in FIG. 5 with a portion of the first laminate layer removed;

FIG. 7 is a cross sectional view of the alternative exemplary patient transfer board taken generally along line 7-7 in FIG. 6;

FIG. 8 is an exploded view of a further alternative exemplary patient transfer board showing an alternative embodiment of a reservoir/pump unit configured for use with a patient transfer board similar to the one shown in FIG. 6;

FIG. 9 a top view of a yet still further alternative exemplary patient transfer board including an embodiment of a heating apparatus and power source;

FIG. 10 is a perspective view of the patient transfer board shown in FIG. 9;

FIG. 11 is a perspective view of the patient transfer board shown in FIG. 9 with certain features shown in phantom lines;

FIG. 12 is an exploded view of the patient transfer board shown in FIG. 9;

FIG. 13 is a rear view of the patient transfer board shown in FIG. 9;

FIG. 14 is a front view of the patient transfer board shown in FIGS. 9; and

FIG. 15 is a cross-sectional view of patient transfer board taken generally along line 15-15 in FIG. 9.

### DETAILED DESCRIPTION OF THE INVENTION

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Certain terminology may be employed in the following description for convenience rather than for any limiting purpose. For example, the terms “forward” and “rearward,” “front” and “rear,” “right” and “left,” “upper” and “lower,” and “top” and “bottom” designate directions in the drawings to which reference is made, with the terms “inward,” “inner,” “interior,” or “inboard” and “outward,” “outer,” “exterior,” or “outboard” referring, respectively, to directions toward and away from the center of the referenced element, the terms “radial” or “horizontal” and “axial” or “vertical” referring, respectively, to directions or planes which are perpendicular, in the case of radial or horizontal, or parallel, in the case of axial or vertical, to the longitudinal central axis of the referenced element, the terms “proximate” and “distal” referring, respectively, to positions or locations that are close or away from a point of reference, and the terms “downstream” and “upstream” referring, respectively, to directions in and opposite that of fluid flow. Terminology of similar import other than the words specifically mentioned above likewise is to be considered as being used for purposes of convenience rather than in any limiting sense.

In the figures, elements having an alphanumeric designation may be referenced herein collectively or in the alternative, as will be apparent from context, by the numeric portion of the designation only. Further, the constituent parts of various elements in the figures may be designated with separate reference numerals which shall be understood to refer to that constituent part of the element and not the element as a whole. General references, along with references to spaces, surfaces, dimensions, and extents, may be designated with arrows. Angles may be designated as “included” as measured relative to surfaces or axes of an element and as defining a space bounded internally within such element therebetween, or otherwise without such designation as being measured relative to surfaces or axes of an element and as defining a space bounded externally by or outside of such element therebetween. Generally, the measures of the angles stated are as determined relative to a common axis, which axis may be transposed in the figures for purposes of convenience in projecting the vertex of an angle defined between the axis and a surface which otherwise does not extend to the axis. The term “axis” may refer to a line or to a transverse plane through such line as will be apparent from context.

Thermal regulation of a patient's temperature following a medical emergency or traumatic event directly impacts patient outcomes as well as the associated hospital costs incurred during the treatment and rehabilitation of the patient. Current temperature management regimes utilize inefficient mechanical means, such as thermal regulating garments or blankets, or invasive medical treatments that may involve inflicting further trauma to the patient. Therefore, it would be desirable to have a system and method for thermally regulating a patient's temperature following a medical emergency or trauma experience to improve treatment outcomes.

Embodiments of the present invention seek to provide a portable temperature regulating stretcher or litter (hereinafter “patient transfer board). Other aspects of the present invention seek to provide patient transfer boards that include temperature regulating apparatus that are configured to emit thermal radiation (i.e., heat) and/or reduce thermal stress (i.e., cool) to a patient's body when lying thereon. Additional aspects of the present invention seek to provide patient transfer boards that employ mechanical and/or electronic solutions to emit thermal radiation and/or reduce thermal stress to a patient's body. Certain aspects of the present invention seek to provide patient transfer boards that are designed for military or civilian use within hospitals and/or during medical transport, such as via ambulances.

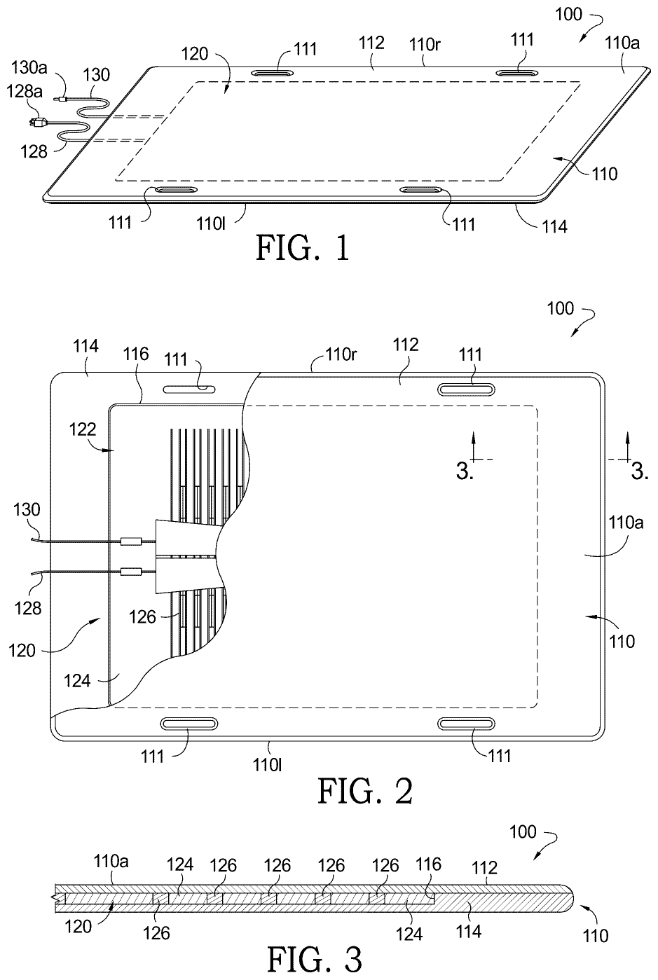

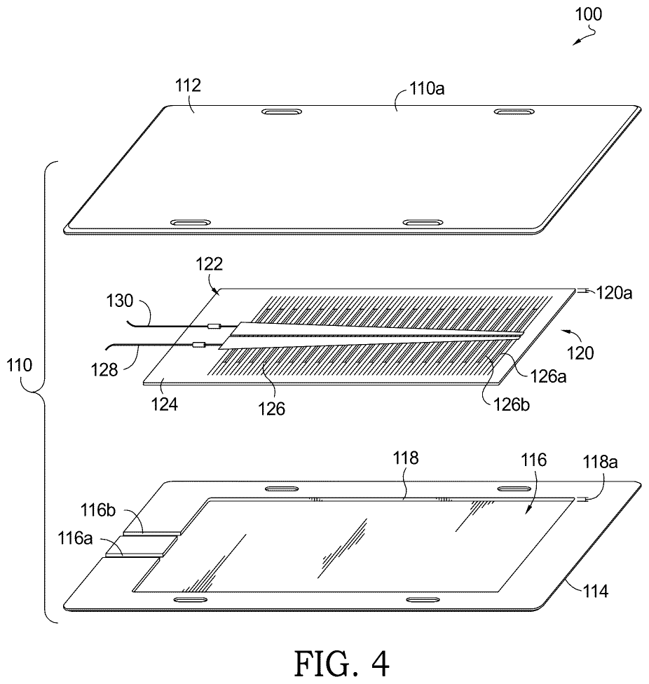

With reference to FIGS. 1-4, an exemplary embodiment of a patient transfer board 100 is shown. As seen more clearly in FIGS. 3 and 4, patient transfer board 100 may include a board body 110 manufactured as a laminate that includes at least two laminate layers, such as a first laminate layer 112 and a second laminate layer 114, using materials such as, but not limited to, carbon fiber, fiber glass, composites, or plastics. In one exemplary embodiment, at least first laminate layer 112 is constructed of carbon fiber material. It should also be noted that while shown and described as having two layers, board body 110 may be rigid and include any number of laminate layers depending upon system requirements and/or end-user preference.

Patient transfer board 100 may further include a temperature regulating apparatus, in the form of a heating unit 120, embedded or disposed between laminate layers 112, 114 and may be selectively powered to emit thermal radiation to patient transfer board 100 (and thus, the patient located thereon) as needed, as will be described in greater detail below. To facilitate transport of patient transfer board 100 when a patient is positioned on patient transfer board 100, board body 110 may include one or more handholds 111 that are peripherally defined therein (e.g., positioned proximate one or both side edges 1101, 110r of board body 110). Users, such as medical personnel, may grasp patient transfer board 100 by inserting their hands through handholds 111 to lift, carry, transport, or otherwise transfer the patient from one location to another. Handholds 111 may also be configured to receive and affix retaining elements (e.g., patient restraint straps) to assist in maintaining the position of the patient on board body 110. In some aspects of the present invention, handholds 111 may traverse laminated layers 112, 114.

As seen most clearly in FIG. 4, exemplary heating unit 120 may include a generally rectangular and flexible heating panel 122, although it should be noted that other geometric shapes/sizes of heating panels may be used depending upon the final structural form of patient transfer board 100. In accordance with an aspect of the present invention, heating panel 122 may include a base member or substrate 124 upon or within which is positioned an array of heating elements 126. For example, and without limitation thereto, heating elements 126 may include resistive ink printed onto base member/substrate 124. Heating elements 126 may be formed in any geometric pattern that results in an operable electrical circuit. Other suitable materials for heating element 126 can include, but is not limited to, resistive wires, coils, strips, or ribbons fabricated from any suitable restrictive material including metals, alloys, ceramics, composites, polymers, carbons, or any combination thereof.

As shown in FIG. 4, heating elements 126 may be formed as a plurality of spatially separated and generally parallel subunits (e.g., heating element subunits 126a, 126b), wherein the plurality of subunits extend substantially the entire length and width of base member/substrate 124 to emit thermal radiation symmetrically. Alternatively, the subunits can be positioned along the length and/or width of base member/substrate 124 to emit thermal radiation asymmetrically. Being formed of a resistive material, heating elements 126 may be configured to emit thermal radiation when supplied with an electrical current. In other words, heating elements 126 may be configured to emit thermal radiation symmetrically or asymmetrically to the surface of patient transfer board 100 when supplied with an electric current.

In one aspect of the present invention, patient transfer board 100 may be configured to be powered through either a wall outlet or through a battery or other portable power source. By way of example and without limitation, and as further shown in FIGS. 1 and 4, base member 124 may include a first power coupling, such as power cord 128 configured to terminate in a two-prong or three-prong outlet plug 128a, for plugging patient transfer board 100 into a standard AC electrical outlet. Base member 124 may additionally or alternatively include a second power coupling, such as power cord 130 which is configured to terminate in a charger plug 130a, such as but not limited to a male USB or micro-USB connector, for plugging patient transfer board 100 into a battery pack or other mating charging port of a portable power supply.

With additional reference to FIG. 2, second laminate layer 114 may be constructed so as to define a recess 116 configured to receive heating unit 120 therein and may further include one or more channels 116a, 116b dimensioned to receive power cords 128, 130, respectively, therein. Recess sidewall 118 may have a depth 118a equal to or slightly less than the thickness 120a of heating unit 120. Following lamination, first laminate layer 112 may then lie flat on second laminate layer 114 so as to produce a generally smooth, planar top surface 110a of board body 110 of patient transfer board 100.

In one aspect of the present invention, heating element 126 may be a positive temperature coefficient (PTC) heating element configured to self-modify so as to output a consistent, user-selected maximum temperature. To reduce the probability of exceeding the thermal limits of heating elements 126 and potentially cause thermal damage to user tissue, the maximum thermal output of heating element 126 may be preselected during the manufacturing phase of heating unit 120. For example, heating element 126 may thus have a predetermined maximum thermal output that allows the surface temperature of planar top surface 110a of patient transfer board 100 to not exceed about 80 degrees Celsius.

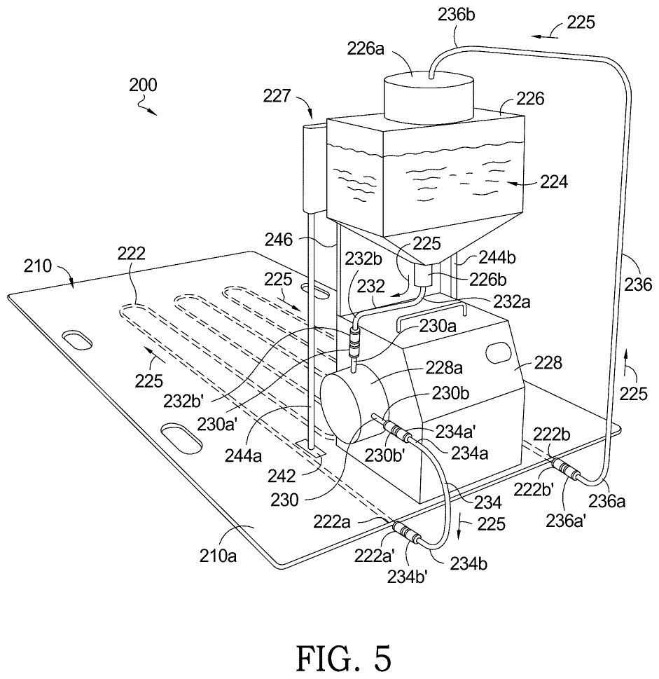

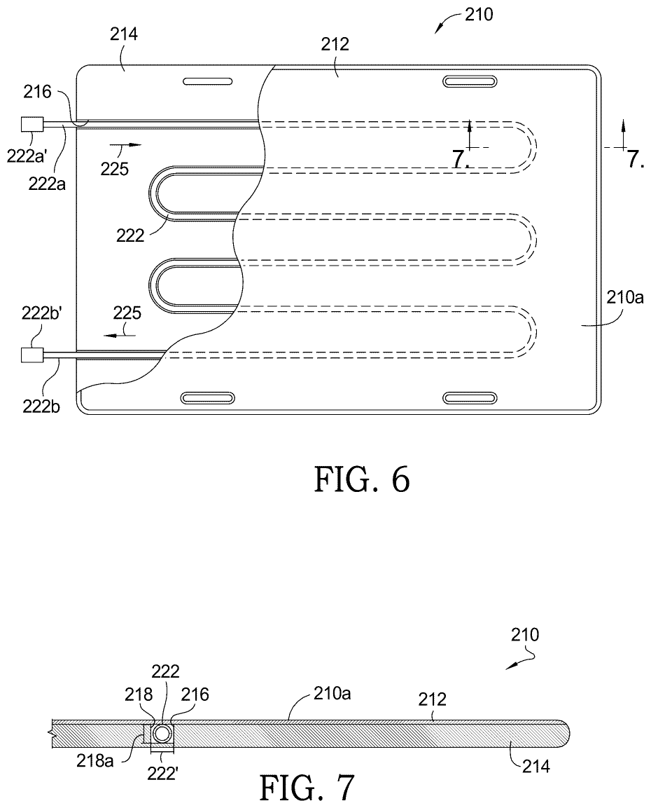

In accordance with another aspect of the present invention, and with specific reference to FIGS. 5-7, an alternative exemplary embodiment of a patient transfer board 200 for thermal regulation is shown. Patient transfer board 200 may include one or more similar components, features, materials, and/or functionalities of patient transfer board 100 described above. Patient transfer board 200 may be configured to selectively emit thermal radiation or reduce thermal stress to a patient, as needed once the patient has been placed atop patient transfer board 200. As seen most clearly in FIGS. 6 and 7, patient transfer board 200 may include a board body 210 manufactured as a laminate similar to patient transfer board body 110. Board body 210 may include at least two laminate layers, such as a first laminate layer 212 and a second laminate layer 214, that may be formed using materials such as, but not limited to, carbon fiber, fiber glass, composites, or plastics. In one exemplary embodiment, at least first laminate layer 212 is constructed of carbon fiber material. It should also be noted that while shown and described as having two layers, board body 210 may include any number of laminate layers depending upon system requirements and/or end-user preference.

As seen most clearly in FIG. 5, an exemplary heating unit 220 may include a fluid tube 222 embedded between laminate layers 212, 214 and may be, for example and without limitation, placed in a serpentine pattern covering a substantial portion of laminate layers 212, 214 so as to provide generally symmetric thermal radiation emissions/thermal stress reductions across the surface of patient transfer board 200. It should be noted that other geometric shapes/sizes/patterns of fluid tube may be used depending upon the desired structure of patient transfer board 200. In accordance with an aspect of the present invention, fluid tube 222 is a made of a flexible material exhibiting good thermal transfer characteristics. Non-limiting examples of fluid tube 222 material may include any suitable TYGON tubing, cross-linked polyethylene (PEX), silicone or polyvinyl chloride (PVC) tubing, and the like. In certain aspects of the present invention, fluid tube 222 can be made of a non-flexible material.

Fluid tube 222 may be configured to selectively provide thermal radiation emissions or thermal stress reduction to patient transfer board 210 when provided with a temperature regulating fluid 224 flowing therethrough. In one aspect of the invention, thermal regulating fluid 224 is water, although any suitable fluid may be used. By way of example and without limitation, reservoir 226 may be filled with ice water to decrease a patient's body temperature, or may be filled with hot water (e.g., between about 70 degrees Celsius and about 80 degrees Celsius) to raise the patient's body temperature. Applicable temperature regulating fluids include, but are not limited to, glycol-water mixtures, mineral oil, silicone oils, synthetic fluids, and similar compositions. As shown in FIG. 5, temperature regulating fluid 224 may be stored within a fluid reservoir 226 and directed through fluid tube 222 via a pump 228 in a recirculating pathway generally indicated by arrows 225. In one aspect of the present invention, pump 228 may be a positive displacement pump such as a diaphragm pump, and more preferably, a peristaltic pump. By way of example, peristaltic pump 228 may include a pump head 228a equipped with a plurality of rollers (not shown) which are rotated to sequentially push fluid from reservoir 226 to board body 210.

In one aspect of the present invention, patient transfer board 200 is designed for military or civilian use within hospitals and/or during medical transport, such as via ambulances. By using peristaltic pump 228, contamination of physical pump components may be minimized because cleaning of the pump's flow path requires only changing of the portion of the pump tubing being used within pump head 228a. For instance, a first tube portion 230 may be located within pump head 228a and include first and second ends 230a, 230b that extends outwardly therefrom, with each terminating in a respective fitting, such as a quick connect coupling 230a′, 230b′. A second tube portion 232 may then include a first end 232a fluidly coupled to outlet end 226b of reservoir 226 and a second end 232b coupled to first end 230a of first tube portion 230, such as via a respective quick connect coupling 232b′ engaged with quick connect coupling 230a′.

Third tube portion 234 may then include a first end 234a fluidly coupled to second end 230b of first tube portion 230 and a second end 234b coupled to an inlet end 222a of fluid tube 222 within board body 210. To assist coupling, a respective quick connect coupling 234b′ on third tube portion 234 may engage with quick connect coupling 222a′ on inlet end 222a of fluid tube 222. As a result, temperature regulating fluid 224 within reservoir 226 may pass sequentially through second tube portion 232, first tube portion 230 and third tube portion 234 into fluid tube 222 to regulate the temperature of body board 210 and thus expose patients to thermal radiation emissions or thermal stress reductions, as needed. Alternatively, third tube portion 234 may be omitted and first tube portion 230 may be of sufficient length whereby quick connect coupling 230b′ at second end 230b may be directly coupled to quick connect coupling 222a′ at inlet end 222a.

Fourth tube portion 236 may then include a first end 236a fluidly coupled to outlet end 222b of fluid tube 222, such as via respective quick connect couplings 236a′ and 222b′, while second end 236b of fourth tube portion 236 is coupled to inlet end 226a of reservoir 226. Thus, temperature regulating fluid 224 exiting fluid tube 222 at outlet end 222b may be recirculated to reservoir 226 to thereby complete recirculating pathway 225.

With additional reference to FIGS. 6 and 7, second laminate layer 214 may be formed to define a recess 216 dimensioned to receive fluid tube 222 therein. Recess sidewall 218 may have a depth 218a equal to or slightly less than the thickness 222′ of fluid tube 222. First laminate layer 212 may then lie flat on second laminate layer 214 so as to produce a generally smooth, planar top surface 210a of patient transfer board 200. In a further aspect of the invention and with reference to FIG. 5, second laminate layer 214 may further define one or more mounting recesses 240 dimensioned to receive respective one or more mounting plates 242 therein. Respective mounting plates 242 (only one shown) may be configured to securely and mechanically couple with respective mounting rods 244a, 244b to thereby mount reservoir 226 onto top surface 210a of patient transfer board 200 during use. During non-patient transport and storage of patient transfer board 200, mounting rods 244a, 244b may be decoupled from their respective mounting plates 242 so as to separate the reservoir/pump unit 227 from the generally planar sheet of board body 210. Reservoir 226 may be further coupled to pump 228 via a support bracket 246 to form reservoir/pump unit 227.

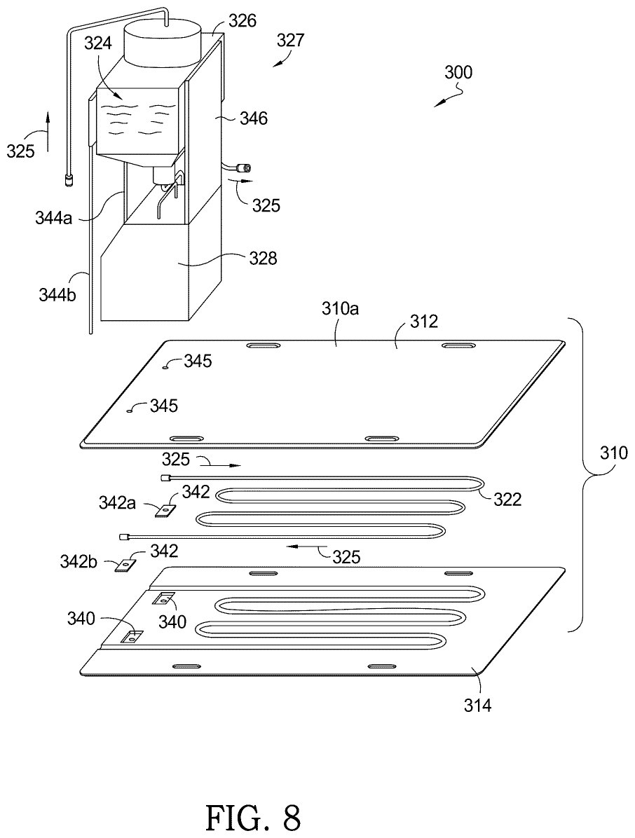

Turning now to FIG. 8, a further alternative exemplary embodiment of a patient transfer board 300 for temperature regulation is shown. Patient transfer board 300 is substantially identical to patient transfer board 200 described above but for an alternative configuration of reservoir/pump unit 327. Patient transfer board 300 may share one or more components, features, materials, and/or functionalities with patient transfer boards 100 or 200. Patient transfer board 300 may include a board body 310 manufactured as a laminate similar to patient transfer board body 110, 210. Board body 310 may include at least two laminate layers, such as a first laminate layer 312 and a second laminate layer 314, and a fluid tube 322 embedded between laminate layers 312, 314. Fluid tube 322 can be configured to be oriented in a serpentine pattern (or other geometric pattern) that covers a substantial portion of laminate layers 312, 314 in a manner to provide generally symmetric thermal radiation emissions/thermal stress reductions across the surface of patient transfer board 300. A temperature regulating fluid 324 may be stored within a fluid reservoir 326 and directed through fluid tube 322 via a pump 328 in a recirculating pathway, generally indicated by arrows 325. Applicable temperature regulating fluids include, but are not limited to, water, glycol-water mixtures, mineral oil, silicone oils, synthetic fluids, and similar compositions.

As similarly described above with regard to patient transfer board 200, second laminate layer 314 of patient transfer board 300 may define one or more mounting recesses 340 dimensioned to receive respective one or more mounting plates 342 therein. Respective mounting plates 342a, 342b may be configured to securely couple with respective mounting rods 344a, 344b to mount reservoir 326 through respective apertures 345 formed in top surface 310a of patient transfer board 300 during use. During non-patient transport and storage, mounting rods 344a, 344b may be decoupled from their respective mounting plates 342a, 342b so as to separate the reservoir/pump unit 327 from the generally planar sheet of board body 310. Reservoir 326 may be further coupled to pump 328 via a support bracket 346 to form reservoir/pump unit 327.

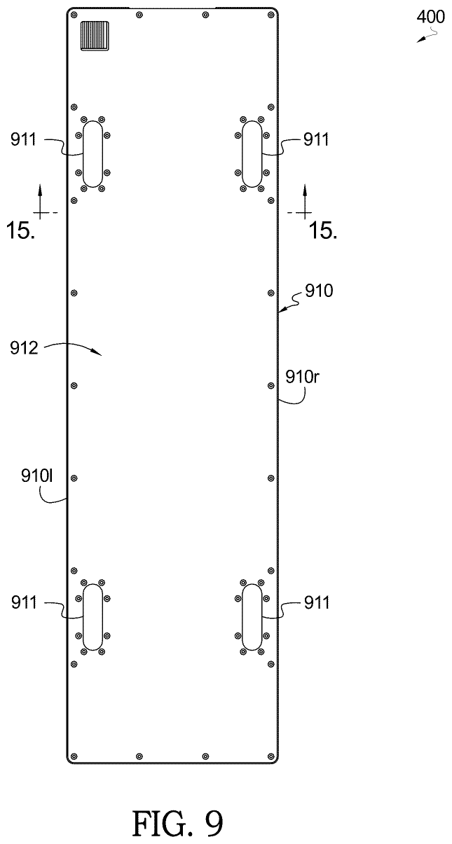

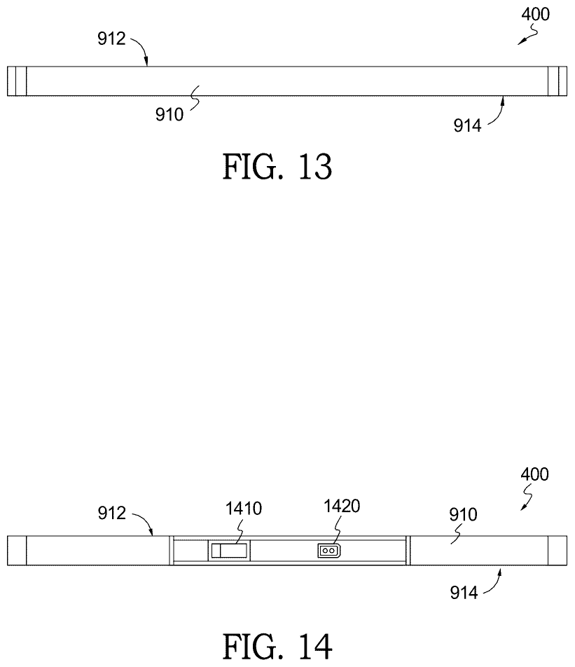

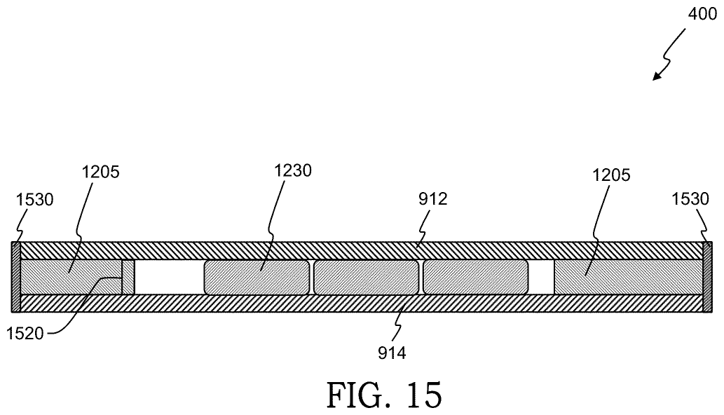

In accordance with another aspect of the present invention, and with specific reference to FIGS. 9-15, a yet still further alternative exemplary embodiment of a patient transfer board 400 for thermal regulation is shown. Patient transfer board 400 may include one or more features, materials, components, functionalities of patient transfer boards 100, 200, and/or 300 described above. In preferred embodiments, patient transfer board 400 may be configured to selectively emit thermal radiation from one or more of its surfaces and thereby increase the core temperature of patients when positioned on or proximate to such surfaces.

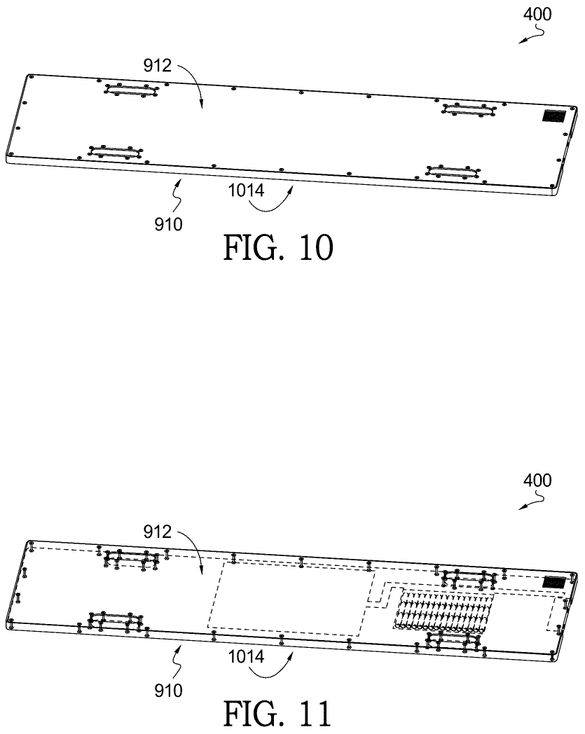

As seen more clearly in FIGS. 10-12, patient transfer board 400 may include a board body 910 manufactured as a laminate including at least two laminate layers, such as a first laminate layer 912 and a second laminate layer 1014, using materials such as, but not limited to, carbon fiber, fiber glass, composites, or plastics. In preferred embodiments, at least one of first laminate layer 912 and second laminate layer 1014 are configured to be at least partially thermally conductive. In one exemplary embodiment, at least first laminate layer 912 is constructed of carbon fiber material. It should also be noted that while shown and described as having two layers, board body 910 may be rigid and include any number of laminate layers depending upon system requirements and/or end-user preference. Board body 910 may further include an intermediate layer 1205 positioned between first laminate 912 and second laminate 1014.

Intermediate layer 1205 may be manufactured using materials such as, but not limited to, foams, composites, or plastics. In some aspects, intermediate layer 1205 may include a thermally conductive material. To assist with the selective emission of thermal radiation, patient transfer board 400 may further include a temperature regulating apparatus in the form of heating unit 1222 and power source 1230, which are conductively coupled to each other and disposed upon or within intermediate layer 1205. Heating unit 1222 may be positioned in recess 1216 defined in intermediate layer 1205. Power source 1230 may be positioned in recess 1217 defined in intermediate layer 1205. In some aspects, recesses 1216, 1217 may be substantially equal to thickness 1520. Heating unit 1222 may be selectively powered to emit thermal radiation to patient transfer board 400 (and thus, the patient located thereon) as needed, as will be described in greater detail below. Power source 1230 may include one or more primary battery cells and/or secondary battery cells. To facilitate transport of patient transfer board 400 when a patient is positioned thereon, board body 910 may include one or more handholds 911 that are peripherally defined therein (e.g., positioned proximate one or both side edges 9101, 910r of board body 910). In accordance with an aspect of the present invention, handholds 911 each traverse board body 910.

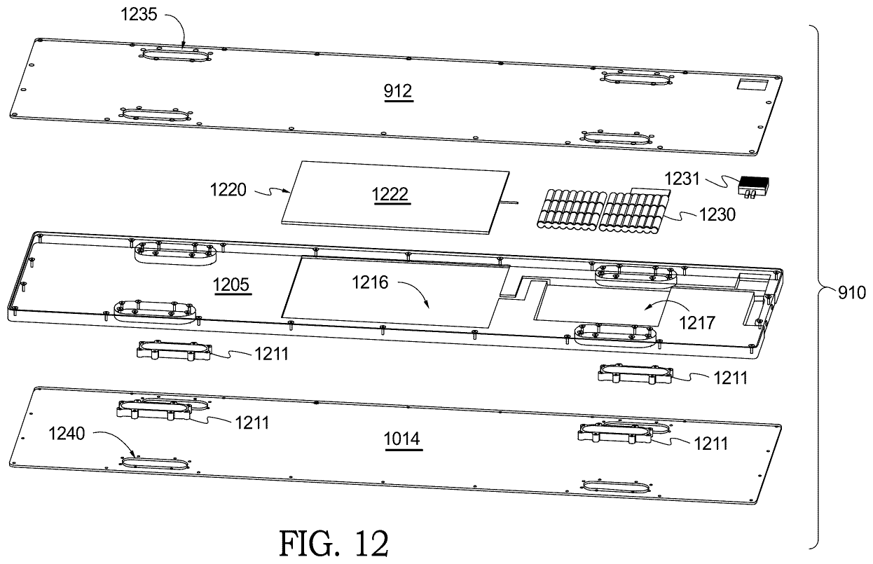

Users, such as medical personnel, may grasp patient transfer board 400 by inserting their hands through handholds 911 to lift, carry, transport, or otherwise transfer the patient from one location to another. Handholds 911 may also be configured to receive and affix retaining elements (e.g., patient restraint straps) to assist in maintaining the position of the patient on board body 910. As seen most clearly in FIG. 12, exemplary heating unit 920 may include a generally rectangular flexible heating panel 922, although it should be noted that other geometric shapes/sizes of heating panels may be used depending upon the final structural form of patient transfer board 400. In accordance with an aspect of the present invention, and similar to heating panel 122, flexible heating panel 922 may include a base member or substrate upon or within which is positioned an array of heating elements. For example, and without limitation thereto, such heating elements may include resistive ink printed onto a base member/substrate. For example, heating unit 920 can share one or more components, materials, features, and/or functionalities of heating unit 120.

Said heating elements may be formed in any geometric pattern that results in an operable electrical circuit. Other suitable materials for the heating elements can include, but are not limited to, resistive wires, coils, strips, or ribbons fabricated from any suitable restrictive material including metals, alloys, ceramics, composites, polymers, carbons, or combination thereof. Such heating elements preferably emit thermal radiation when supplied with an electric current (e.g., provided by power source 1230) and can be configured to emit thermal radiation symmetrically or asymmetrically to the surface of patient transfer board 400. By way of example and without limitation, and as further shown in FIG. 14, board body 910 may include switch 1410 and/or mating charging port 1420. Switch 1410 may be a mechanical or electrical device that is conductively coupled to heating unit 1220 and power source 1230 in a manner to allow a user to selectively supply power to heating panel 1222.

In one aspect of the present invention, mating charging port 1420 may be conductively coupled to power source 1230 and configured to receive a power coupling, such as a power cord that terminates in a multi-prong outlet plug for plugging patient transfer board 400 into a standard AC electrical outlet or charger plug, such as but not limited to, a male USB or micro-USB connector, for plugging patient transfer board 400 into a portable power supply.

The foregoing description of the preferred embodiment of the invention has been presented for the purpose of illustration and description. It is not intended to be exhaustive nor is it intended to limit the invention to the precise form disclosed. It will be apparent to those skilled in the art that the disclosed embodiments may be modified in light of the above teachings. The embodiments described are chosen to provide an illustration of principles of the invention and its practical application to enable thereby one of ordinary skill in the art to utilize the invention in various embodiments and with various modifications as are suited to the particular use contemplated. Therefore, the foregoing description is to be considered exemplary, rather than limiting, and the true scope of the invention is that described in the following claims.

What is claimed is:

1. A board for thermally regulating the temperature of a patient disposed on the board, the board comprising:
a board body including at least two laminate layers, wherein the at least two layers include a first laminate layer and a second laminate layer; and
a temperature regulating apparatus embedded between the first laminate layer and the second laminate layer.

2. The board of claim 1, wherein the temperature regulating apparatus includes a heating element, wherein the heating element selectively emits thermal radiation to at least one of the first laminate layer and the second laminate layer.

3. The board of claim 1, wherein the temperature regulating apparatus includes a fluid tube having a first end and a second end, wherein the fluid tube is configured to receive a flow of a temperature regulating fluid therethrough.

4. The board of claim 3, further comprising a fluid pump and a fluid tank configured for holding the temperature regulating fluid therein, wherein the fluid pump generates the flow of the temperature regulating fluid though the fluid tube.

5. The board of claim 4, wherein the fluid tank, the fluid pump and the fluid tube form a recirculating flow circuit for the temperature regulating fluid.

6. The board of claim 1, wherein the second laminate layer defines a recess dimensioned to receive the temperature regulating apparatus therein.

7. The board of claim 2, wherein the heating element emits thermal radiation when supplied with an electric current.

8. The board of claim 2, wherein the heating element comprises a resistive material.

9. The board of claim 8, wherein the resistive material comprises a resistive ink.

10. The board of claim 2, wherein
the heating element comprises a power coupling; and
the board selectively and conductively couples to a power source via the power coupling.

11. The board of claim 2, further comprising:
an intermediate layer positioned between the first laminate layer and the second laminate layer;
a power source conductively coupled to the heating element; and
wherein the heating element and the power source are positioned upon or within the intermediate layer.

12. The board of claim 1, wherein
the board body comprises a plurality of handholds peripherally defined in the board body; and
the plurality of handholds each traverse the board body.

13. The board of claim 2, wherein
the temperature regulating apparatus comprises a substrate; and
the heating element is positioned upon or within the substrate.

14. A board for thermally regulating the temperature of a patient disposed on the board, the board comprising:
a board body including at least two laminate layers, wherein the at least two layers comprise a first laminate layer and a second laminate layer; and
a temperature regulating apparatus embedded between the first laminate layer and the second laminate layer, wherein the temperature regulating apparatus comprises a heating element, and wherein the heating element selectively heats at least one of the first laminate layer and the second laminate layer.

15. The board of claim 14, further comprising:
a fluid pump; and
a fluid tank fluidly coupled to the fluid pump,
wherein the temperature regulating apparatus comprises a fluid tube having a first end and a second end, wherein the fluid tube is configured to receive a flow of a temperature regulating fluid therethrough;
wherein the fluid tank is configured to hold the temperature regulating fluid therein, and
wherein the fluid pump is configured to generate the flow of the temperature regulating fluid though the fluid tube.

16. The board of claim 15, wherein the fluid tank, the fluid pump and the fluid tube form a recirculating flow circuit for the temperature regulating fluid.

17. A board for thermally regulating the temperature of a patient disposed on the board, the board comprising:
a board body including at least two laminate layers, wherein the at least two layers include a first laminate layer and a second laminate layer;
a temperature regulating apparatus embedded between the first laminate layer and the second laminate layer, wherein the temperature regulating apparatus comprises a heating element, wherein the heating element comprises a resistive material, and wherein the heating element is configured for:
selectively heating at least one of the first laminate layer and the second laminate layer, and
emitting thermal radiation when supplied with an electric current.

18. The board of claim 17, wherein the resistive material comprises a resistive ink.

19. The board of claim 18, further comprising:
an intermediate layer positioned between the first laminate layer and the second laminate layer; and
a power source conductively coupled to the heating element, wherein the heating element and the power source are positioned upon or within the intermediate layer.

20. The board of claim 19, wherein the temperature regulating apparatus comprises a substrate, and wherein the heating element is positioned upon or within the substrate.
